(19) United States
(12) Patent Application Publication
(10) Pub. No.: US 2026/0355083 A1
(43) Pub. Date: Nov. 12, 2026

(54) RINSE-OFF HAIR COSMETIC COMPOSITIONS, METHOD OF TREATING HAIR, METHOD FOR PROVIDING IMPROVED CURLS DEFINITION TO THE HAIR AND USE OF A COMPOSITION

(71) Applicant: L'OREAL, Paris (FR)

(72) Inventors: Cyrus ANDRIOLO, Rio de Janeiro (BR); Érika Alegrio Jarque PETALI, Rio de Janeiro (BR)

(73) Assignee: L'OREAL, Paris (FR)

(21) Appl. No.: 19/487,395

(22) PCT Filed: May 31, 2023
(86) PCT No.: PCT/BR2023/050174

§ 371 Date: Nov. 24, 2025

(51) Int. Cl. A61K 8/44; A61K 8/34; A61K 8/365; A61K 8/37; A61K 8/41; A61K 8/92; A61K 8/9789; A61Q 5/12

(52) U.S. Cl. CPC A61K 8/44; A61K 8/342; A61K 8/365; A61K 8/37; A61K 8/416; A61K 8/922; A61K 8/9789; A61Q 5/12; A61K 2800/43

(57) ABSTRACT

The present invention is directed to rinse-off hair cosmetic compositions, comprising at least one organic acid, a non-aqueous solvent comprising at least one compound with at least one hydroxyl-group, at least one amino acid, at least one fatty compound selected from vegetal oils, vegetal resins, vegetal waxes, and mixtures thereof, water, and at least one surfactant selected from cationic surfactants, nonionic surfactants, and mixtures thereof. The invention is also related to a method of treating hair, particularly curly hair, a method for providing improved curls definition to the hair and a use of a composition for the manufacture of a rinse-off hair care product for providing improved curls definition.

### FIELD OF THE INVENTION

The present invention is directed to rinse-off hair cosmetic compositions comprising (a) at least one organic acid, (b) a non-aqueous solvent comprising at least one compound with at least one hydroxyl-group, (c) at least one amino acid, (d) at least one fatty compound selected from vegetal oils, vegetal resins, vegetal waxes, and mixtures thereof, (e) water, and (f) at least one surfactant selected from cationic surfactants, nonionic surfactants, and mixtures thereof. The invention is also related to a method of treating hair, particularly curly hair, a method for providing improved curls definition to the hair and a use of a composition for the manufacture of a rinse-off hair care product for providing improved curls definition.

### BACKGROUND OF THE INVENTION

People have been using several cosmetic and personal care compositions to enhance the look and feel of keratin fibers, such as the hair.

Particularly regarding curly hair, consumers are always dealing with problems related to the definition of the curls such as frizz, volume, and shape. Curly hair tends to become frizzy and significantly expand in volume when exposed to high humidity conditions, chemical treatments, among others. Additionally, the shape and definition of the curls are lost after sleeping.

Many attempts to developed products to discipline, define and control the hair, in particular curly hair, enhancing/maintaining curl definition, curl retention, and curl regularity, without leaving the hair feeling and/or looking unnatural and stiff were made, but such products resulted in additional problems or maintenance difficulty.

For example, the products of the state of the art usually require high amount of product to give good combing and hair feel. But, considering that the effect of such products is not long lasting, the consumers have to reapply the product several times during the day or after sleeping in order to regain definition and frizz control, resulting in the build-up of the product on the hair's surface, resulting in a heavy aspect, wet look, unnatural touch and greasy sensation, since such products do not penetrate the keratin fibers and remain on the surface of the hair fiber.

In this sense, there is a need in the market for curly hair products that define the curls in a lasting way, without the necessity of reapplying the product until the next wash or after sleeping, also decreasing the need to use large amounts of the product between the washes.

Additionally, the standard curly hair routine includes the use of several cosmetic products through the entire length of the hair and must be reset every two days, since the curls tend to lose definition and fresh-looking aspect after such period—it is a very time-consuming routine that involves the consumers restarting all the steps in about every two days, including washing the hair and applying different cosmetic products.

Therefore, it is desirable to have a rinse-off hair cosmetic product for curly hair that not only helps defining the curls, but also provide long lasting cosmetic benefits to the consumer's hair such as resistance to frizz, that leave the hair shiny and that is easy to apply and does not leave the hair feeling wet or heavy, that improve with the use of such product. There is also the need for the rinse-off hair cosmetic product for curly hair to reduce the quantity of products used by the consumer, and to prolong the effects of the composition so the consumer may gain extra days before having to restart the curly hair cosmetic routine.

The inventors of the present invention surprisingly and unexpectedly achieved rinse-off hair cosmetic compositions, particularly designed for curly hair, comprising (a) at least one organic acid, (b) a non-aqueous solvent comprising at least one compound with at least one hydroxyl-group, (c) at least one amino acid, (d) at least one fatty compound selected from vegetal oils, vegetal resins, vegetal waxes, and mixtures thereof, (e) water and (f) at least one surfactant selected from cationic surfactants, nonionic surfactants, and mixtures thereof, that are able to not only penetrate the hair fiber without building-up on the fiber's surface, while also providing curl definition until the next wash, long lasting of definition of the curls, frizz control, natural look and desirable volume, and that does not look heavy on the hair, but also provides cosmetic benefits such shine, gliding touch, spreadability.

### SUMMARY OF THE INVENTION

The present invention relates to rinse-off hair cosmetic compositions comprising (a) at least one organic acid, (b) a non-aqueous solvent comprising at least one compound with at least one hydroxyl-group, (c) at least one amino acid, (d) at least one fatty compound selected from vegetal oils, vegetal resins, vegetal waxes, and mixtures thereof, (e) water, and (f) at least on surfactant selected from cationic surfactants, nonionic surfactants, and mixtures thereof. The present invention is also directed to a method for treating hair, particularly curly hair, comprising the step of contacting the hair with the rinse-off hair cosmetic composition of the invention. The present invention also relates to a method for providing improved curls definition to the hair, comprising contacting the hair with the rinse-off hair cosmetic composition of the invention. The present invention also relates to a use of a composition for the manufacture of a rinse-off hair care product for providing improved curls definition.

### BRIEF DESCRIPTION OF THE DRAWINGS

FIG. 1 relates to the results of comparative In Vitro Test 1 wherein the rinse-off hair cosmetic compositions of the present invention (Ex. 1 and Ex. 2) were used in the routine as conditioner compared with standard conditioner in the market in view of definition.

FIG. 2 relates to the results of the In Vitro Test 2 wherein the rinse-off hair cosmetic composition of the present invention (Ex. 1) was used in the routine as mask with different times between application of the composition to the hair and removal from the hair compared with standard mask in the market in view of definition (FIG. 2A) and volume and frizz control (FIG. 2B).

FIG. 3 relates to the results of the In Vitro Test 3 wherein the rinse-off hair cosmetic composition of the present invention (Ex. 2) was used in the routine as mask with different times between application of the composition to the hair and removal from the hair compared with standard mask in the market in view of definition (FIG. 3A) and volume and frizz control (FIG. 3B).

### DETAILED DESCRIPTION OF THE INVENTION

The present invention is directed to a rinse-off hair cosmetic composition, mainly for curly hair, comprising:
(a) at least one organic acid;
(b) a non-aqueous solvent comprising at least one compound with at least one hydroxyl-group;
(c) at least one amino acid;
(d) at least one fatty compound selected from vegetal oils, vegetal resins, vegetal waxes, and mixtures thereof;
(e) water; and
(f) at least on surfactant selected from cationic surfactants, nonionic surfactants, and mixtures thereof.

In an embodiment, the at least one organic acid is selected from short chain organic acids, preferably α-hydroxy acids (AHAs), beta hydroxy acids, and a mixture thereof. In an embodiment, the at least one organic acid of the rinse-off hair cosmetic composition of the present invention is selected from tartaric acid, lactic acid, glutaric acid, etidronic acid, salicylic acid, glycolic acid, citric acid, and mixtures thereof.

In a preferred embodiment, the at least one organic acid is tartaric acid.

In a preferred embodiment, the at least one organic acid is lactic acid.

In an embodiment, the at least one organic acid includes tartaric acid and a second organic acid selected from lactic acid, glutaric acid, etidronic acid, salicylic acid, glycolic acid, and citric acid.

In a preferred embodiment, the at least one organic acid includes tartaric acid and lactic acid.

Also, the amount of the at least one organic acid ranges from about 0.1% to about 25.0% by weight, 0.5% to about 20.0% by weight, preferably from about 0.5% to about 10.0% by weight, more preferably from about 0.75% to about 6.0% by weight, even more preferably from about 1.0% to about 5.0%, and most preferably from about 1.0% to about 4.0%, based on the total weight of the composition.

The rinse-off hair cosmetic composition according to the present invention also comprises a non-aqueous solvent comprising at least one compound with at least one hydroxyl-group. Without being limited to any one theory, it is believed that the non-aqueous solvent comprising at least one compound with at least one hydroxyl-group acts as a penetrating agent, that is, being capable of forming the dipole-dipole interaction with the keratin fibers, such as hair, and binding to polar sites on the surface of said keratin fiber and decrease the shielding, thus promoting penetration of the hair cosmetic composition into the keratin fibers.

In an embodiment, the non-aqueous solvent comprising at least one compound with at least one hydroxyl-group of the rinse-off hair cosmetic composition of the present invention is selected from polyols, C₂ to C₆ monoalcohols, and a mixture thereof.

In an embodiment, the non-aqueous solvent comprising at least one compound with at least one hydroxyl-group of the rinse-off hair cosmetic composition of the present invention is selected from polyols. In an embodiment, the polyol is a glycol selected from propylene glycol, dipropylene glycol, butylene glycol, pentylene glycol, hexylene glycol, glycerin, and mixtures thereof. In an embodiment, the polyol comprises propylene glycol and dipropylene glycol. In an embodiment, the polyol is glycerin. In an embodiment, the polyol comprises propylene glycol and glycerin.

In an embodiment, the non-aqueous solvent comprising at least one compound with at least one hydroxyl-group of the rinse-off hair cosmetic composition of the present invention is selected from C₂ to C₆ monoalcohols. In an embodiment, the C₂ to C₆ monoalcohols include ethanol and/or propanol and/or isopropyl alcohol (isopropanol) and/or butanol and/or hexanol.

In an embodiment, the non-aqueous solvent comprising at least one compound with at least one hydroxyl-group of the rinse-off hair cosmetic composition of the present invention comprises a mixture of polyols and C₂ to C₆ monoalcohols. In an embodiment, the non-aqueous solvent comprising at least one compound with at least one hydroxyl-group of the rinse-off hair cosmetic composition of the present invention comprises propylene glycol and ethanol. In an embodiment, the non-aqueous solvent comprising at least one compound with at least one hydroxyl-group of the rinse-off hair cosmetic composition of the present invention comprises propylene glycol and isopropyl alcohol. In an embodiment, the non-aqueous solvent comprising at least one compound with at least one hydroxyl-group of the rinse-off hair cosmetic composition of the present invention comprises propylene glycol, isopropyl alcohol, and ethanol. In an embodiment, the non-aqueous solvent comprising at least one compound with at least one hydroxyl-group of the rinse-off hair cosmetic composition of the present invention comprises propylene glycol, isopropyl alcohol, ethanol, and glycerin. In an embodiment, the non-aqueous solvent comprising at least one compound with at least one hydroxyl-group of the rinse-off hair cosmetic composition of the present invention comprises propylene glycol, ethanol, and glycerin.

The amount of the non-aqueous solvent comprising at least one compound with at least one hydroxyl-group ranges from about 0.1% to about 20.0% by weight, preferably from about 0.3% to about 15.0% by weight, or from about 0.5% to about 12.0% by weight, or from about 0.5% to about 11.0% by weight, or from about 1.0% to about 11.0% by weight, based on the total weight of the composition.

According to the present invention, the at least one amino acid is selected from arginine and glycine.

In an embodiment, the at least one amino acid is selected from arginine.

In an embodiment, the at least one amino acid is selected from glycine.

In an embodiment, the at least one amino acid is selected from arginine and glycine.

The amount of the at least one amino acid ranges from about 0.1% to about 7.0% by weight, preferably from about 0.1% to about 6.0% by weight, more preferably from about 0.1% to about 5.0% by weight, even more preferably from about 0.5% to about 3.0% weight, based on the total weight of the composition. Other suitable ranges are from about 0.5% to about 2.0% by weight, or from about 0.7% to about 1.5% by weight, based on the total weight of the composition.

In an embodiment, the at least one fatty compound selected from vegetal oils, vegetal resins, vegetal waxes, and mixtures thereof is selected from Copaiba officinalis (Balsam Copaiba) resin (oil resin or resin form).

In a further embodiment, the at least one fatty compound selected from vegetal oils, vegetal resins, vegetal waxes, and mixtures thereof is selected from coconut oil, Soja Soybean oil (soybean oil) and mixtures thereof.

In an embodiment, the at least one fatty compound includes coconut oil.

In an embodiment, the at least one fatty compound includes Soja Soybean oil.

In an embodiment, the at least one fatty compound selected from vegetal oils, vegetal resins, vegetal waxes, and mixtures thereof includes Copaiba Resin, Candeia resin, and white breu resin.

In an embodiment, the at least one fatty compound selected from vegetal oils, vegetal resins, vegetal waxes, and mixtures thereof is Copaiba resin.

In an embodiment, the at least one fatty compound selected from vegetal oils, vegetal resins, vegetal waxes, and mixtures thereof includes Copaiba resin and coconut oil, and Soja Soybean oil.

In an embodiment, the at least one fatty compound selected from vegetal oils, vegetal resins, vegetal waxes, and mixtures thereof includes Copaiba resin and coconut oil.

In an embodiment, the at least one fatty compound selected from vegetal oils, vegetal resins, vegetal waxes, and mixtures thereof includes Candelilla wax.

In another embodiment, the at least one fatty compound selected from vegetal oils, vegetal resins, vegetal waxes, and mixtures thereof includes Carnauba wax.

The amount of the at least one fatty compound selected from vegetal oils, vegetal resins, vegetal waxes, and mixtures thereof ranges from about 0.1% to about 15.0% by weight, preferably from about 0.1% to about 14.0% by weight, preferably from about 0.15% to about 10.0% by weight, preferably from about 0.17% to about 8.0% by weight, or such as at about 0.1%, about 0.17%, about 0.2%, about 0.3%, about 0.36%, about 0.4%, about 0.5% to about 6.0% by weight, based on the total weight of the composition.

In an embodiment, the at least one surfactant selected from cationic surfactants, nonionic surfactants, and mixtures thereof includes at least one cationic surfactant. In a preferred embodiment, the at least one cationic surfactant is selected from quaternary ammonium salts such as cetrimonium chloride, stearimonium chloride, behentrimonium chloride, behentrimonium methosulfate, behenamidopropyltrimonium methosulfate, stearamidopropyltrimonium chloride, arachidtrimonium chloride, distearyldimonium chloride, dicetyldimonium chloride, tricetylmonium chloride, and from cationizable surfactants, for example, amido amine compounds such as oleamidopropyl dimethylamine, linoleamidopropyl dimethylamine, isostearamidopropyl dimethylamine, stearamidopropyl dimethylamine, behenamidopropyl dimethylamine, behenamidopropyldiethylamine, behenamidoethyldiethyl-amine, behenamidoethyldimethylamine, arachidamidopropyldimethylamine, arachidamido-propyidiethylamine, arachidamidoethyidiethylamine, arachidamidoethyidimethylamine, brassicamidopropyldimethylamine, lauramidopropyl dimethylamine, myristamidopropyl dimethylamine, dilinoleamidopropyl dimethylamine, palmitamidopropyl dimethylamine, dipalmitoylethyl hydroxyethylmonium methosulfate, and mixtures thereof.

In an embodiment, the at least one surfactant selected from cationic surfactants, nonionic surfactants, and mixtures thereof includes at least one cationic surfactant selected from behentrimonium chloride, cetrimonium chloride, behentrimonium methosulfate, and mixtures thereof.

In an embodiment, the at least one surfactant selected from cationic surfactants, nonionic surfactants, and mixtures thereof includes at least one cationic surfactant selected from stearamidopropyl dimethylamine, brassicamidopropyl dimethylamine, and mixtures thereof.

In an embodiment, the at least one surfactant selected from cationic surfactants, nonionic surfactants, and mixtures thereof includes at least one nonionic surfactant. In a preferred embodiment, the at least one nonionic surfactant is selected from fatty alcohols.

In an embodiment, the fatty alcohols include cetyl alcohol, cetearyl alcohol, stearyl alcohol, lauryl alcohol, myristyl alcohol, arachidyl alcohol, behenyl alcohol, and mixtures thereof.

In an embodiment, the fatty alcohols include cetearyl alcohol.

In an embodiment, the fatty alcohols include cetyl alcohol.

In an embodiment, the fatty alcohols include stearyl alcohol.

In an embodiment, the fatty alcohols include cetearyl alcohol and stearyl alcohol.

In an embodiment, the fatty alcohols include an alkoxylated alcohol.

In an embodiment, the fatty alcohols include an alkoxylated alcohol selected from steareth-20, Laureth-12, ceteareth-20, deceth-10, trideceth-6, and mixtures thereof.

In an embodiment, the at least one nonionic surfactant is selected from at least one glucoside. In a preferred embodiment, the at least one glucoside is selected from arachidyl glucoside, coco-glucoside, caprylyl/capryl glucoside and mixtures thereof.

In an embodiment, the least one glucoside of the rinse-off hair cosmetic composition of the present invention ranges from about 0 to about 5%, from about 0% to about 3%, from about 0.1% to about 3% by weight based on the total weight of the composition.

In an embodiment, the at least one nonionic surfactant is selected from at least one fatty acid. In a preferred embodiment, the at least one fatty acid is selected from stearic acid, myristic acid, oleic acid, and mixtures thereof.

In an embodiment, the least one fatty acid of the rinse-off hair cosmetic composition of the present invention ranges from about 0 to about 5%, from about 0% to about 2%, from about 0.1% to about 2% by weight based on the total weight of the composition.

In an embodiment, the at least one nonionic surfactant is selected from at least one monoglyceride. In a preferred embodiment, the at least one monoglyceride is selected from glyceryl stearate, glyceryl oleate, and mixtures thereof.

In an embodiment, the least one monoglyceride of the rinse-off hair cosmetic composition of the present invention ranges from about 0 to about 5%, from about 0% to about 3%, from about 0.1% to about 3% by weight based on the total weight of the composition.

In an embodiment, the at least one surfactant includes at least one cationic surfactant selected from behentrimonium chloride, cetrimonium chloride, behentrimonium methosulfate, stearamidopropyl dimethylamine, brassicamidopropyl dimethylamine, dipalmitoylethyl hydroxyethylmonium methosulfate, and mixtures thereof, and at least one nonionic surfactant selected from cetyl alcohol, cetearyl alcohol, stearyl alcohol, lauryl alcohol, myristyl alcohol, arachidyl alcohol, behenyl alcohol, and mixtures thereof.

When the at least one surfactant includes an amido amine such as stearamidopropyl dimethylamine or brassicamidopropyl dimethylamine, a pH adjuster agent such as triethanolamine may be included in the composition.

The amount of the at least one surfactant selected from cationic surfactants, nonionic surfactants, and mixtures thereof ranges from about 0.05% to about 15.0% by weight, preferably from about 1.0% to about 14.0% by weight, preferably from about 1.5% to about 12.0% by weight, more preferably from about 1% to about 12% by weight, from about 2.0% to about 11.0%, from about 3.0% to about 10.0% by weight, including all ranges and subranges, based on the total weight of the rinse-off hair cosmetic composition.

Individually, each surfactant may be present in an amount such as about 0.05%, about 0.5%, about 1.0%, about 1.5%, about 1.8%, about 2.4%, about 3.0%, about 3.5%, about 4.0%, about 5.0%, about 5.5%, about 6.0%, about 6.5%, about 7.0, about 7.5%, about 8.0%, about 8.5%, about 9.0%, about 9.5%, about 10. 0%, about 10.5%, about 11.0%, about 11.5%, about 12.0% by weight, based on the total weight of the composition.

In a preferred embodiment, the at least one surfactant is at least one cationic surfactant ranging from about 0.1% to about 10.0% by weight, such as from about 1.0% to about 10.0% by weight, from about 1.4% to about 9.0% by weight, from about 1.4% to about 8.0% by weight, from about 1.4% to about 6.0% by weight, from about 2.0 to about 6.0% by weight, from about 2.4 to about 6.0% by weight, based on the total weight of the composition.

In a preferred embodiment, the at least one surfactant is at least one nonionic surfactant selected from at least one fatty alcohol ranging from about 0.1 to about 12.0% by weight, such as from about 3.0 to about 12.0% by weight, from about 4.0% to about 12.0% by weight, from about 5.0% to about 10.0% by weight, from about 6.0% to about 9.0% by weight based on the total weight of the composition.

In an embodiment, the rinse-off hair cosmetic composition of the present invention further comprises at least one ester, which may be selected from isopropyl palmitate, dicaprylyl carbonate, cetyl esters, and mixtures thereof.

In an embodiment, the rinse-off composition of the invention comprises one or more pigments. In a preferred embodiment, the pigment includes violet pigment.

In an embodiment, the pigment of the rinse-off hair cosmetic composition of the present invention ranges from about 0 to about 1% by weight based on the total weight of the composition. In a preferred embodiment, the pigment of the rinse-off hair cosmetic composition of the present invention ranges from about 0.1% to 0.5% by weight based on the total weight of the composition.

In an embodiment, the rinse-off composition of the invention is in the form of a conditioner or a masque (mask). In a preferred embodiment, the rinse-off composition is in the form of a conditioner. In a preferred embodiment, the rinse-off composition is in the form of a mask.

In various embodiments, the hair cosmetic composition of the present invention is a conditioner composition in the form of an aqueous composition or an emulsion or a cream or a lotion.

In a preferred embodiment, the rinse-off hair cosmetic composition of the present invention is in the form of an emulsion (oil in water).

In other various embodiments, the hair cosmetic composition of the present invention is a masque (mask) composition in the form of a thick or highly viscous emulsion or cream or lotion.

In a further preferred embodiment, the rinse-off hair cosmetic composition can be, but not exclusive, free of or substantially free of silicon, behentrimonium chloride, and/or a quaternary ammonium salt.

In an embodiment, the rinse-off hair cosmetic compositions according to the invention are free of or substantially free of anionic surfactants.

The pH of the rinse-off hair cosmetic composition of the present invention is from about 1 to about 10, preferably from about 2 to about 7, or from about 2 to about 6. To achieve lasting of definition, the pH is about 3 to about 5, preferably from about 2.5 to about 4.5, or from about 3.0 to about 4.5, or from about 3.5 to about 4.5, or from about 3.0 to about 4.0, or at about 2, about, 3, about 3.3, about 3.5, about 4, about 4.5.

Moreover, the present invention is directed to a method for treating hair, particularly curly hair, comprising the step of (1) contacting the hair with the rinse-off hair cosmetic composition of the invention and the step of (2) rising-off the composition from the hair.

In a preferred embodiment, the period of time between the steps (1) and (2), that is, the time between application of the composition to the hair and removal from the hair, ranges from about 0 to about 30 minutes. In an embodiment, the time between application of the composition to the hair and removal from the hair is about 30 minutes. In an embodiment, the time between application of the composition to the hair and removal from the hair is about 15 minutes. In an embodiment, the time between application of the composition to the hair and removal from the hair is about 10 minutes. In an embodiment, the time between application of the composition to the hair and removal from the hair is about 5 minutes. In another embodiment, the time between application of the composition to the hair and removal from the hair is about 3 minutes. In a further embodiment, the time between application of the composition to the hair and removal from the hair is about 1 minute. In an embodiment, there is no time between application of the composition to the hair and removal from the hair.

Additionally, the present invention also relates to a method for providing improved curls definition to the hair, comprising contacting the hair with the rinse-off hair cosmetic composition of the invention.

The present invention also relates to a use of a composition for the manufacture of a rinse-off hair care product for providing improved curls definition.

In a preferred embodiment, the composition of the invention is used as a rinse-off product to deliver long lasting definition of curls without build up and frizz, that improve curl regularity, discipline, volume control, individualization of the curls, and dries fast, among other attributes.

### Organic Acid

The at least one organic acid of the compositions can be selected from alpha hydroxy acids, beta hydroxy acids, and a mixture thereof.

The term “alpha-hydroxy acid” is understood to mean, according to the present invention, a carboxylic acid having at least one hydroxyl functional group occupying an alpha-position on said acid (carbon adjacent to a carboxylic acid functional group). A beta-hydroxy acid is closely related to an alpha-hydroxy acid except the carboxylic and hydroxyl functional groups are separated by two carbon atoms. A hydroxy acid can be present in the final composition in the form of the free acid and/or in the form of one of its associated salts (salts with an organic base or an alkali metal, in particular), especially according to the final pH imposed on the composition.

The alpha hydroxy acid present in the composition, according to the disclosure, includes, but is not limited to, one or more of tartaric acid, glycolic acid, citric acid, lactic acid, methyllactic acid, glucuronic acid, pyruvic acid, 2-hydroxybutanoic acid, 2-hydroxypentanoic acid, 2-hydroxyhexanoic acid, 2-hydroxyheptanoic acid, 2-hydroxyoctanoic acid, 2-hydroxynonanoic acid, 2-hydroxydecanoic acid, 2-hydroxyundecanoic acid, 2-hydroxydodecanoic acid, 2-hydroxytetradecanoic acid, 2-hydroxyhexadecanoic acid, 2-hydroxyoctadecanoic acid, 2-hydroxytetracosanoic acid, 2-hydroxyeicosanoic acid, mandelic acid, phenyllactic acid, gluconic acid, galacturonic acid, aleuritic acid, ribonic acid, tartronic acid, malic acid, fumaric acid, their salts and their mixtures. It is also possible to use mixtures of these various acids.

In some embodiments, the composition includes alpha hydroxy acids comprising tartaric acid and lactic acid. When combined, the more than one alpha hydroxy acids may be present in the same or in different amounts.

The beta hydroxy acid, when present in the composition according to the disclosure, includes, but is not limited to, one or more of salicylic acid and derivatives thereof (including 5-n-octanoylsalicylic acid, salicylate, sodium salicylate, and willow extract), beta hydroxybutanoic acid, tropic acid, and trethocanic acid.

In some embodiments, the composition includes tartaric acid.

In some embodiments, the composition includes tartaric acid and lactic acid.

### Compound with at Least One Hydroxyl-Group

The non-aqueous solvent of the present invention comprises at least one compound with at least one hydroxyl-group.

Said compound with at least one hydroxyl-group can be selected from polyols, and/or C₂ to C₆ monoalcohols such as ethyl alcohol, isopropyl alcohol, propyl alcohol, ethylene glycol, propylene glycol, butylene glycol, hexylene glycol, propane diol, and glycerin.

Further non-limiting examples include alkanediols (polyhydric alcohols) such as glycerin, 1,2,6-hexanetriol, trimethylolpropane, ethylene glycol, propylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, caprylyl glycol, 1,2-hexanediol, 1,2-pentanediol, and 4-methyl-1,2-pentanediol; alkyl alcohols having 1 to 4 carbon atoms such as ethanol, methanol, butanol, propanol, isopropanol, and a mixture thereof.

In some cases, the compound with at least one hydroxyl-group may be selected from the group consisting of one or more glycols, C1-4 alcohols, glycerin, and a mixture thereof. In some cases, the compound with at least one hydroxyl-group is selected from the group consisting of hexylene glycol, propylene glycol, dipropylene glycol, caprylyl glycol, glycerin, isopropyl alcohol, and a mixture thereof.

Polyhydric alcohols are useful. Examples of polyhydric alcohols include glycerin, ethylene glycol, diethylene glycol, triethylene glycol, propylene glycol, dipropylene glycol, tripropylene glycol, 1,3-butanediol, 2,3-butanediol, 1,4-butanediol, 3-methyl-1,3-butanediol, 1,5-pentanediol, tetraethylene glycol, 1,6-hexanediol, 2-methyl-2,4-pentanediol, polyethylene glycol, 1,2,4-butanetriol, 1,2,6-hexanetriol, and a mixture thereof.

Polyol compounds may also be used. Non-limiting examples include the aliphatic diols, such as 2-ethyl-2-methyl-1,3-propanediol, 3,3-dimethyl-1,2-butanediol, 2,2-diethyl-1,3-propanediol, 2-methyl-2-propyl-1,3-propanediol, 2,4-dimethyl-2,4-pentanediol, 2,5-dimethyl-2,5-hexanediol, 5-hexene-1,2-diol, and 2-ethyl-1,3-hexanediol, and a mixture thereof.

### Amino Acid

The compositions according to the disclosure comprise at least one amino acid. As used herein, the term “amino acid” includes amino acids such as proteinogenic amino acids, amino sulfonic acids, and salts thereof.

Amino acids are simple organic compounds containing both a carboxylic acid group (—COOH) and an amino group (—NH₂). Amino sulfonic acids are simple organic compounds containing both a sulfonic acid group (—SO₂OH) and an amino group (—NH₂). Accordingly, the amino acids useful according to the disclosure may, in certain embodiments, be selected from compounds of Formula (I) and Formula (II):

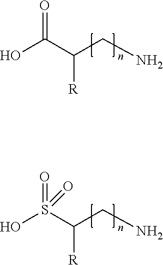

wherein:
R represents a hydrogen atom, a linear or branched, preferably linear, C₁-C₅ alkyl group, said alkyl group being optionally substituted with at least one group chosen from hydroxyl, —C(O)—OH, —S(O)₂—OH, —C(O)—O⁻ and M⁺, and S(O)₂—O⁻ and M⁺, with M⁺ representing a cationic counter-ion such as an alkali metal, alkaline earth metal, or ammonium, and
n is 0 or 1.

The amino acids may be in their non-ionized form (I) and (II) or in their ionized or betaine form (I′) and (II′):

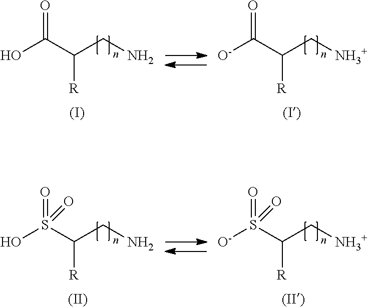

wherein R and n are as defined above.

The one or more amino acids may also be in their conjugate base form (Ib) and (IIb):

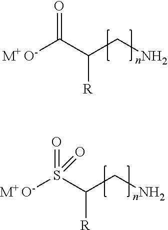

wherein R and n are as defined above.

Well-known amino acids include the twenty amino acids that form the proteins of living organisms (standard proteinogenic amino acids): alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine. The amino acids of the instant disclosure, however, are not limited to the standard proteinogenic amino acids.

Non-limiting examples of amino sulfonic acids include aminomethane sulfonic acid, 2-aminoethane sulfonic acid (taurine), aminopropane sulfonic acid, aminobutane sulfonic acid, aminohexane sulfonic acid, aminoisopropyl sulfonic acid, aminododecyl sulfonic acid, aminobenzene sulfonic acid, aminotoulene sulfonic acid, sulfanilic acid, chlorosulfanilic acid, diamino benzene sulfonic acid, amino phenol sulfonic acid, amino propyl benzene sulfonic acid, amino hexyl benzene sulfonic acid, and a mixture thereof.

In some cases, charged amino acids may be used. Non-limiting examples of charged amino acids include arginine, lysine, aspartic acid, and glutamic acid. In some cases, polar amino acids are useful. Non-limiting examples of polar amino acids include glutamine, asparagine, histidine, serine, threonine, tyrosine, cysteine, methionine, and tryptophan.

In some cases, hydrophobic amino acids may be employed. Non-limiting examples of hydrophobic amino acids include alanine, isoleucine, leucine, phenylalanine, valine, proline, and glycine.

### Fatty Compound

The at least one fatty compound of the rinse-off hair cosmetic composition of the present invention may be selected from vegetal oils, vegetal resins, vegetal waxes, and mixtures thereof.

### Vegetal Oil

In an embodiment, the fatty compound of the rinse-off hair cosmetic composition of the present invention selected from vegetal oils, vegetal resins, vegetal waxes, and mixtures thereof comprises at least one vegetal oil.

In a non-limitative and preferred embodiment, the at least one vegetal oil is selected from coconut oil, almond oil, aloe vera oil, avocado oil, soybean oil, camelina oil, coffee oil, castor oil, grapeseed oil, jojoba oil, basil oil, bay oil, bergamot oil, black pepper essential oil, birch oil, camphor, Cannabis oil, caraway oil, cardamom seed oil, carrot seed oil, chamomile oil, calamus root oil, cinnamon oil, citronella oil, clary sage, clove leaf oil, coffee, coriander, costmary oil, cranberry seed oil, cubeb, cumin oil, dill oil, Eucalyptus oil, fennel seed oil, fenugreek oil, fir, frankincense oil, geranium oil, ginger oil, grapefruit oil, grapeseed oil, henna oil, horseradish oil, jasmine oil, juniper berry oil, lavender oil, lemon oil, lemongrass, marjoram, melaleuca, lemon balm oil, mountain savory, mugwort oil, mustard oil, myrrh oil, myrtle, neem tree oil, neroli, nutmeg, orange oil, oregano oil, orris oil, palo santo, parsley oil, peppermint oil, pine oil, plum oil, rose oil, rosehip oil, rosemary oil, rosewood oil, sandalwood oil, spruce, star anise oil, tarragon oil, tea tree oil, thyme oil, and mixtures thereof.

In some embodiments, the suitable vegetal oils for use in the hair care compositions of the present invention are selected from coconut oil, soybean oil, canola oil, rapeseed oil, corn oil, cottonseed oil, olive oil, palm oil, peanut oil, safflower oil, sesame oil, sunflower oil, linseed oil, palm kernel oil, wheatgerm oil, apricot kernel oil, pistachio oil, poppy oil, hazel nut oil, colza oil, cade oil, peach kernel oil, jojoba oil, Helianthus annuus (sunflower) seed oil, and mixtures thereof.

When present, the vegetal oil can improve the penetration effect.

In a preferred embodiment, the vegetal oil of the present invention is coconut oil, that is mainly composed by Lauric acid, Myristic Acid, Palmitic Acid, Oleic Acid, Caprylic Acid, Capric Acid. Other oils with the same composition could apply on the invention, such as Babassu oil.

In another preferred embodiment, the rinse-off hair cosmetic composition of the present invention further comprises at least one vegetal oil selected from, argan oil, soybean oil and mixtures thereof. In a preferred embodiment, the rinse-off hair cosmetic compositions of the present invention comprise a mixture of vegetal oils and vegetal resins, such as coconut oil and Copaiba officinalis (Balsam Copaiba resin) (Copaiba resin or Copaiba oleoresin).

### Vegetal Resin

In an embodiment, the fatty compound selected from vegetal oils, vegetal resins, vegetal waxes, and mixtures thereof of the rinse-off hair cosmetic composition of the present invention comprises at least one vegetal resin.

Preferably, the vegetal resin of the present invention is Copaifera officinalis (BALSAM Copaiba) RESIN (INCI name) which can be in the form of a Copaiba oil resin or Copaiba resin. Copaifera officinalis (BALSAM Copaiba) RESIN is commercially available under the tradename of RAIN FOREST RF3310 (also known by the chemical name of Copaiba Oleoresin) or under the tradename of RAIN FOREST 03370 (also known by the chemical name of Copaiba resin), both from the supplier Clariant.

### Vegetal Waxes

In an embodiment, the fatty compound selected from vegetal oils, vegetal resins, vegetal waxes, and mixtures thereof comprises a vegetal wax.

When present, the vegetal wax may be selected from Candelilla wax, Carnauba wax, beeswax, and mixtures thereof.

### Water

The rinse-off hair cosmetic compositions according to the present invention comprise water. The water used may be sterile demineralized water and/or deionized water and/or a floral water such as rose water, cornflower water, chamomile water or lime water, and/or a natural thermal or mineral water such as, for example: water from Vittel, water from the Vichy basin, water from Uriage, water from La Roche Posay, water from La Bourboule, water from Enghien-les-Bains, water from Saint Gervais-les-Bains, water from Neris-les-Bains, water from Allevar-les-Bains, water from Digne, water from Maizieres, water from Neyrac-les-Bains, water from Lons-le-Saunier, water from EauxBonnes, water from Rochefort, water from Saint Christau, water from Les Fumades, water from Tercis-les-Bains or water from Avene. Water may also comprise reconstituted thermal water, that is to say a water comprising trace elements such as zinc, copper, magnesium, etc., reconstituting the characteristics of a thermal water.

The rinse-off hair cosmetic composition according to the present invention may comprise water in an amount ranging from about 30.0% to about 99.0% by weight, preferably from about 65.0% to about 95.0% by weight, and most preferably from about 70.0% to about 92.0% by weight, relative to the total weight of the rinse-off hair cosmetic composition.

### Esters

In preferential embodiments, the rinse-off hair cosmetic composition of the present invention comprises at least one ester. The at least one ester may be selected from, but not limited to, isopropyl palmitate, dicaprylyl carbonate, cetyl esters, and mixtures thereof.

When present, the amount of the at least one ester ranges from about 0.5% to about 10.0% by weight, from about 1.5% to about 5.0% by weight, from about 0.5% to about 2.5% by weight, from about 1.0% to about 3.0% by weight, from about 2.0% to about 5.0% by weight, from about 2.0% to about 3.0% by weight, from about 5.0% to about 8.5% by weight, including all ranges and subranges, based on the total weight of the composition.

### Additional Ingredients

In addition to the essential components described hereinbefore, the hair composition of the present invention may further comprise any usual cosmetically acceptable ingredient, which may be chosen especially from such as cosmetically acceptable ingredients selected from perfume/fragrance, preserving agents, vitamins, vegetal extracts, pH-adjusters, and mixtures thereof. Non-limiting examples of preservative agents that can be used according to the invention include salicylic acid, chlorhexidine dihydrochloride, phenoxyethanol and sodium benzoate.

A person skilled in the art will take care to select the optional additional ingredients and/or the amount thereof such that the advantageous properties of the composition according to the invention are not, or are not substantially, adversely affected by the envisaged addition.

The additional ingredients may represent from about 1% to about 70% by weight, based on the total weight of the composition.

### TERMS

As used herein, “rinse-off” composition refers to a composition that is rinsed and/or washed with water either after or during the application of the composition onto the hair, and before drying and/or styling said hair. At least a portion of the composition is removed from the hair during the rinsing and/or washing.

“Conditioner” as used herein means a product/composition capable of imparting to one or more hair fibers at least one property chosen from combability, moisture-retentivity, luster, shine, and softness. The state of conditioning can be evaluated by any means known in the art, such as, for example, measuring, and comparing, the ease of combability of the treated hair and of the untreated hair in terms of combing work (gm-in), and consumer perception.

The term “treat” (and its grammatical variations) as used herein refers to the application of the compositions of the present disclosure onto the surface of hair.

The term “treat” (and its grammatical variations) as used herein also refers to contacting hair with the compositions of the present disclosure.

As used herein, the expression “at least one” means one or more and thus includes individual components as well as mixtures/combinations.

Other than in the operating examples, or where otherwise indicated, all numbers expressing quantities of ingredients and/or reaction conditions are to be understood as being modified in all instances by the term “about,” meaning within +/−5% of the indicated number.

As used herein, all ranges provided are meant to include every specific range within, and combination of sub ranges between, the given ranges. Thus, a range from 1-5, includes specifically 1, 2, 3, 4 and 5, as well as sub ranges such as 2-5, 3-5, 2-3, 2-4, 1-4, etc. All ranges and values disclosed herein are inclusive and combinable. For examples, any value or point described herein that falls within a range described herein can serve as a minimum or maximum value to derive a sub-range, etc.

The term “substantially free” or “essentially free” as used herein means that there is less than about 5% by weight of a specific material added to a composition, based on the total weight of the compositions. Nonetheless, the compositions may include less than about 2 wt. %, less than about 1 wt. %, less than about 0.5 wt. %, less than about 0.1 wt. %, less than 0.01 wt. %, or none of the specified material.

The term “active material” as used herein with respect to the percent amount of an ingredient or raw material, refers to 100% activity of the ingredient or raw material.

“Cosmetically acceptable” means that the item in question is compatible with a keratinous substrate such as skin and hair. For example, a “cosmetically acceptable carrier” means a carrier that is compatible with a keratinous substrate such as skin and hair.

The term “hair cosmetic composition” as used herein can also be a hair styling or a hair shaping or a hair care or a hair treatment composition.

The term “cationic surfactant” means a surfactant that is positively charged. This surfactant may bear one or more positive permanent charges or may contain one or more functions that are cationizable in the composition according to the disclosure.

Throughout the disclosure, the term “a combination thereof” (or a mixture thereof) may be used following a list of elements as shown in the following example where letters A-F represent the elements: “one or more elements selected from the group consisting of A, B, C, D, E, F, and a combination thereof.” The term, “a combination thereof” does not require that the combination include all of A, B, C, D, E, and F (although all of A, B, C, D, E, and F may be included). Rather, it indicates that a combination of any two or more of A, B, C, D, E, and F can be included. In other words, it is equivalent to the phrase “one or more elements selected from the group consisting of A, B, C, D, E, F, and a combination of any two or more of A, B, C, D, E, and F.”

Likewise, the term “a salt thereof” also relates to “salts thereof.” Thus, where the disclosure refers to “an element selected from the group consisting of A, B, C, D, E, F, a salt thereof, and a combination thereof,” it indicates that that one or more of A, B, C, D, and F may be included, one or more of a salt of A, a salt of B, a salt of C, a salt of D, a salt of E, and a salt of F may be included, or a combination of any two of A, B, C, D, E, F, a salt of A, a salt of B, a salt of C, a salt of D, a salt of E, and a salt of F may be included.

The salts referred to throughout the disclosure may include salts having a counter-ion such as an alkali metal, alkaline earth metal, or ammonium counter-ion.

This list of counter-ions, however, is non-limiting.

Some of the compounds discussed throughout the disclosure may be in the form of a salt in the composition or added to the composition in the form of a salt (and dissociate in the composition). Thus, all compounds and amounts of compounds relate to both the salt form of the compound and to the disassociated form of the compound. In other words, even if the expression “a salt thereof” is not specifically or expressly stated with respect to ingredients that can form salts or are available as salts, it is understood that the salt form of the compound is included.

The expression “inclusive” for a range of concentrations means that the limits of the range are included in the defined interval.

Other than in the operating examples, or where otherwise indicated, all numbers expressing quantities of ingredients and/or reaction conditions are to be understood as being modified in all instances by the term “about,” meaning within +/−5% of the indicated number.

The term “INCI” is an abbreviation of International Nomenclature of Cosmetic Ingredients, which is a system of names provided by the International Nomenclature Committee of the Personal Care Products Council to describe personal care ingredients.

All components and elements positively set forth in this disclosure can be negatively excluded from the claims. In other words, the compositions of the instant disclosure can be free or essentially free of all components and elements positively recited throughout the instant disclosure.

Some of the various categories of components identified may overlap. In such cases where overlap may exist and the composition includes both components (or the composition includes more than two components that overlap), an overlapping component or ingredient does not represent more than one component. For example, if a polyacrylate falls within the description of a thickening polymer and within the description of film forming polymer, the specifically recited polyacrylate is understood as being only the thickening polymer or only the film forming polymer. A specific polyacrylate cannot simultaneously be construed as both a thickening polymer and a film forming polymer, for example, when the claims set forth both a thickening polymer and a film forming polymer (even though the polyacrylate may function as both a thickening polymer and a film forming polymer). In other words, a single compound or ingredient cannot simultaneously serve as two different components of a claimed composition.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

### Process for Manufacturing a Rinse-Off Hair Cosmetic Composition

The hair care composition of the present invention can be manufactured from conventional cosmetic processes. An illustrative process comprises the following steps:
Step (1): in a vessel, adding from 30% to 70% of the water and the at least one organic acid, and mixing for about 10 minutes at room temperature;
Step (2): adding the at least one amino acid, mixing the ingredients until complete solubilization;
Step (3): adding the non-aqueous solvent comprising at least one compound with at least one hydroxyl-group, at least one fatty compound selected from vegetal oils, vegetal resins, vegetal waxes, and mixtures thereof and the at least one surfactant selected from cationic surfactants, nonionic surfactants, and mixtures thereof;
Step (4): heating the mixture until about 60 °C to about 75 °C for about 15 minutes;
Step (5): adding the remaining water into the mixture, and cooling down;
Step (6): adding additional ingredients (if present) cooling down until about 25 °C, adjusting the pH (if necessary) with a pH adjuster.

### Embodiments

An example of a suitable hair cosmetic composition according to the present invention comprises: In a preferred embodiment, the hair cosmetic composition of the present invention comprises:
(a) from about 0.5% to about 8.0% by weight of at least one organic acid selected from tartaric acid, lactic acid, glutaric acid, etidronic acid, salicylic acid, and a mixture thereof, preferably from tartaric acid, lactic acid, and mixtures thereof;
(b) from about 0.1% to about 20.0% by weight of non-aqueous solvent comprising at least one compound with at least one hydroxyl-group, said compound selected from polyols, C₂ to C₆ monoalcohols, and a mixture thereof;
(c) from about 0.1% to about 7.0% by weight of arginine;
(d) from about 0.1% to about 7.0% by weight of at least one fatty compound selected from vegetal oils, vegetal resins, vegetal waxes, and mixtures thereof;
(e) water; and
(f) from about 0.5% to about 7.0% by weight of a cationic surfactant selected from behentrimonium chloride, brassicamidopropyl dimethylamine, stearamidopropyl dimethylamine, and mixtures thereof and from about 6.0% to about 11.0% by weight of a nonionic surfactant selected from cetearyl alcohol, stearyl alcohol, and mixtures thereof.

Another preferred embodiment of the hair cosmetic composition of the present invention comprises:
(a) from about 0.5% to about 8.0% by weight of at least one organic acid selected from tartaric acid, lactic acid, glutaric acid, etidronic acid, salicylic acid, and a mixture thereof, preferably from tartaric acid, lactic acid, and mixtures thereof;
(b) from about 0.1% to about 20.0% by weight of a non-aqueous solvent comprising at least one compound with at least one hydroxyl-group, said compound selected from propylene glycol, C₂ to C₆ monoalcohols, glycerin, and a mixture thereof;
(c) from about 0.1% to about 7.0% by weight of arginine;
(d) from about 0.1% to about 7.0% by weight of Copaiba officinalis (Balsam Copaiba) resin;
(e) water,
(f) from about 0.5% to about 7.0% by weight of a cationic surfactant selected from behentrimonium chloride, brassicamidopropyl dimethylamine, stearamidopropyl dimethylamine, and mixtures thereof and from about 6.0% to about 11.0% by weight of a nonionic surfactant selected from cetearyl alcohol, stearyl alcohol, and mixtures thereof; and
(g) from about 1.0% to about 5.0% by weight of at least one vegetal oil selected from coconut oil.

In some embodiments, the compositions of the present disclosure include from about 0.5% to about 1.2% by weight of glycine.

In some embodiments, the compositions of the present disclosure include from about 0.5% to about 5% by weight of esters selected from isopropyl esters (for example, isopropyl palmitate), dialkyl esters (for example, dicaprylyl carbonate), cetyl esters, and mixtures thereof.

In a further embodiment, the hair cosmetic composition of the present invention comprises:
(a) from about 0.5% to about 2.0% by weight of tartaric acid and lactic acid;
(b) from about 0.4% to about 3.5% by weight of propylene glycol, isopropyl alcohol and glycerin;
(c) from about 0.75% to about 2.5% by weight of arginine and glycine;
(d) from about 4.0% to about 6.0% by weight of Copaifera officinalis (balsam Copaiba) resin and Cocos nucifera (coconut) oil;
(e) water;
(f) from about 5.0% to about 9.0% by weight of cetearyl alcohol and behentrimonium chloride;
(g) from about 1.0% to about 3.0% by weight of dicaprylyl carbonate and cetyl esters.

An additional suitable embodiment of the hair cosmetic composition of the present invention comprises:
(a) from about 2.0% to about 4.0% by weight of tartaric acid;
(b) from about 4.5% to about 9.5% by weight of propylene glycol, ethanol, isopropyl alcohol and cetearyl alcohol;
(c) from about 0.5% to about 2.0% by weight of glycine;
(d) from about 3.0% to about 5.0% by weight of Candelilla wax;
(e) water;
(f) from about 1.5% to about 2.5% by weight of behentrimonium chloride;
(g) from about 2.0% to about 5.5% by weight of isopropyl palmitate, dicaprylyl carbonate and cetyl esters.

### Examples

By way of non-limiting illustration, the invention will now be described with reference to the following examples.

Suitable rinse-off hair cosmetic compositions according to the present invention are as Examples 1 to 2, as follows:

| Function/Type | Ingredient | Ex. 1 | Ex. 2 |
|---|---|---|---|
| Amino Acid | arginine | 1.5 | 1.5 |
| Organic Acid | tartaric acid | 1.5 | 1.5 |
| Organic Acid | lactic acid | 0.5 | 0.5 |
| Vegetal resin | Copaifera officinalis | 0.3 | 0.3 |
| | (balsam copaiba) resin | | |
| Vegetal Oil | Cocos nucifera | 4.5 | 4.5 |
| | (coconut) oil | | |
| Non-Aqueous Solvent | propylene glycol | 1.3 | 1.3 |
| Surfactant | cetearyl alcohol | 6.5 | 6.5 |
| Surfactant | behentrimonium chloride | 3.5 | 3.5 |
| Ester | dicaprylyl carbonate | 1.5 | 1.5 |
| Ester | cetyl esters | 1.5 | 1.5 |
| Miscellaneous ingredients/ | one or more of | </=2.0 | </=2.0 |
| Additives | fragrance, preservatives, | | |
| | vitamins | | |
| pH adjuster | triethanolamine | 0.5 | 0.5 |
| Pigment | Violet extract | — | 0.2 |
| Aqueous Solvent | water | Q.S. | Q.S. |

The amounts are described on percentage by weight, based on the total weight of the composition.

### Test 1—In Vitro Tests of Definition and Volume & Frizz Control on Hair Swatches—Conditioner with and without Pigment

The rinse-off hair cosmetic compositions according to Examples 1 and 2 of the present invention were compared to a state-of-the-art conditioner composition comprising water, cetearyl alcohol, dimethicone, glycerin, behentrimonium chloride, fragrance, quaternium-33, lactic acid, hydroxycitronellal, phenoxyethanol, behentrimonium methosulphate, limonene, benzyl salicylate, linalool, benzyl alcohol, amodimethicone, isopropyl alcohol, 2-oleamido-1,3-octadecanediol, cetrimonium chloride, citronellol, cetyl alcohol, coumarin and hexyl cinnamal and amyl cinnamal.

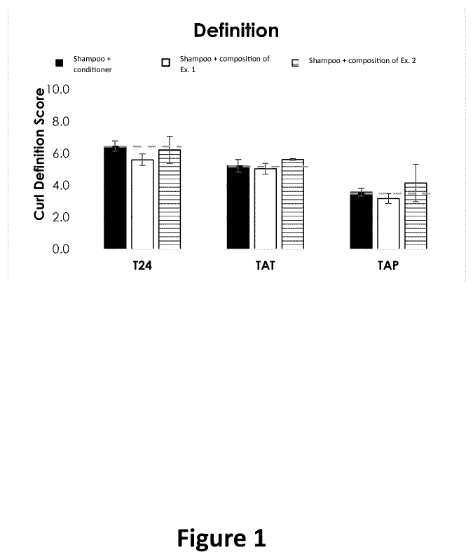

Compositions of Ex. 1 and 2 and the state-of-the-art composition were applied on virgin curly hair swatches (three 2.7-gram swatches per composition) after the step of contacting the hair with a shampoo, and were assessed according to a curl definition scoring (from 0 to 10) at 24 hours after treating the hair (T24), at 24 hours but after breaking the rigid effect (passing the fingers through the hair (TAT) and after combing the swatch of TAT (TAP). The definition measurement was done by imaging data. The results are presented on FIG. 1.

Additionally, compositions of Ex. 1 and 2 and the state-of-the-art composition were applied on virgin curly hair swatches (three 2.7-gram swatches per composition) after the step of contacting the hair with a shampoo and were assessed according to volume and frizz control. The measurement data was generated by a trained panel and the score goes from 1 to 4 in 0.5 in 0.5 where 4 is the highest score (best value).

Particularly, data of FIG. 1 shows that when substitute a market conditioner by the composition Ex. 1, the definition has similar performance. However, when putting the Violet pigment, composition Ex. 2, the definition shows tendency of superiority into definition (T24) and lasting definition (TAT and TAP).

### Test 2—In Vitro Tests of Definition and Volume & Frizz Control on Hair Swatches—Mask

The rinse-off hair cosmetic composition according to Example 1 of the present invention was compared to a state-of-the-art mask composition comprising water, cetearyl alcohol, dimethicone, glycerin, behentrimonium chloride, fragrance, lactic acid, hydroxycitronellal, sodium hydroxide, hydroxypropyltrimonium hydrolyzed wheat protein, hydrolyzed rice protein, phenoxyethanol, arginine, limonene, benzyl salicylate, linalool, benzyl alcohol, pentylene glycol, amodimethicone, isopropyl alcohol, 2-oleamido-1,3-octadecanediol, serine, cetrimonium chloride, citronellol, coumarin, hexyl cinnamal and amyl cinnamal.

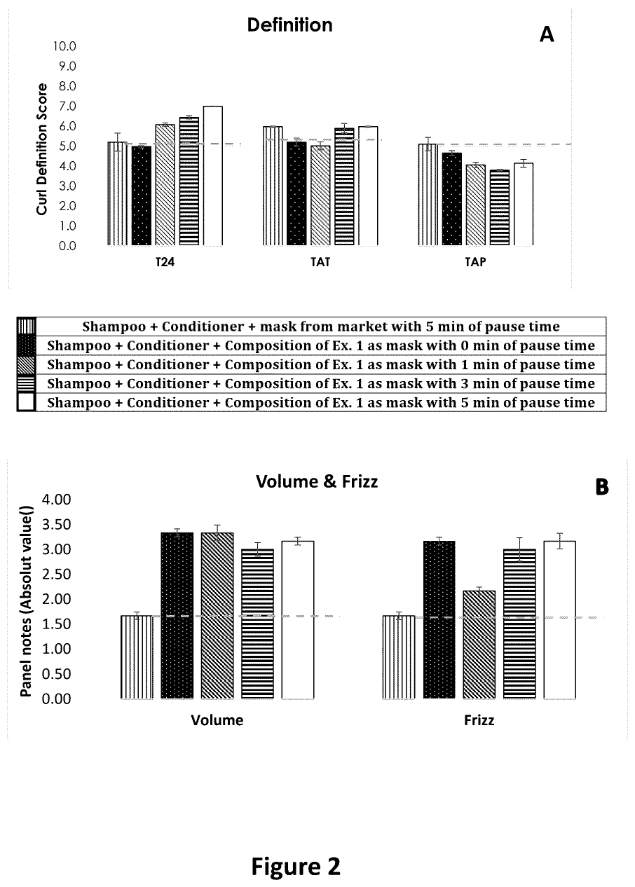

Composition of Ex. 1 and the state-of-the-art composition were applied on virgin curly hair swatches (three 2.7-gram swatches per composition) after the steps of contacting the hair with a shampoo and a conditioner, and were assessed according to a curl definition scoring (from 0 to 10) at 24 hours after treating the hair (T24), at 24 hours but after breaking the rigid effect (passing the fingers through the hair (TAT) and after combing the swatch of TAT (TAP). The definition measurement was done by imaging data. The results are presented on FIG. 2A for several times between application to the hair and removal from the hair including no time between application of the composition to the hair and removal from the hair (0 min.), 1 minute between application of the composition to the hair and removal from the hair (1 min.), 3 minutes between application of the composition to the hair and removal from the hair (3 min.), and 5 minutes between application of the composition to the hair and removal from the hair (5 min.).

Additionally, composition of Ex. 1 and the state-of-the-art were applied on virgin curly hair swatches (three 2.7-gram swatches per composition) after the steps of contacting the hair with a shampoo and a conditioner and were assessed according to volume and frizz control. The measurement data was generated by a trained panel and the score goes from 1 to 4 in 0.5 in 0.5 where 4 is the highest score (best value). The results are presented on FIG. 2B.

As showed from the FIGS. 2A and 2B, the rinse-off hair care composition of Ex. 1 of the present invention used as mask has superior results in volume and frizz control in any time between application of the composition to the hair and removal from the hair (0, 1, 3 or 5 min) then reference mask with 5 min between application of the composition to the hair and removal from the hair. In definition performance, the composition of Ex.1 of the present invention, when used as mask with no time (application followed by rinse), already has equal definition as reference with 5 min between application of the composition to the hair and removal from the hair. Furthermore, when it comes to higher times between application of the composition to the hair and removal from the hair (1, 3 or 5 min), the composition of Ex. 1 of the present invention shows definition superiority.

### Test 3—In Vitro Tests of Definition and Volume & Frizz Control on Hair Swatches—Mask with Pigment

The rinse-off hair cosmetic composition according to Example 2 of the present invention was compared to a state-of-the-art mask composition comprising water, cetearyl alcohol, dimethicone, glycerin, behentrimonium chloride, fragrance, lactic acid, hydroxycitronellal, sodium hydroxide, hydroxypropyltrimonium hydrolyzed wheat protein, hydrolyzed rice protein, phenoxyethanol, arginine, limonene, benzyl salicylate, linalool, benzyl alcohol, pentylene glycol, amodimethicone, isopropyl alcohol, 2-oleamido-1,3-octadecanediol, serine, cetrimonium chloride, citronellol, coumarin, hexyl cinnamal and amyl cinnamal.

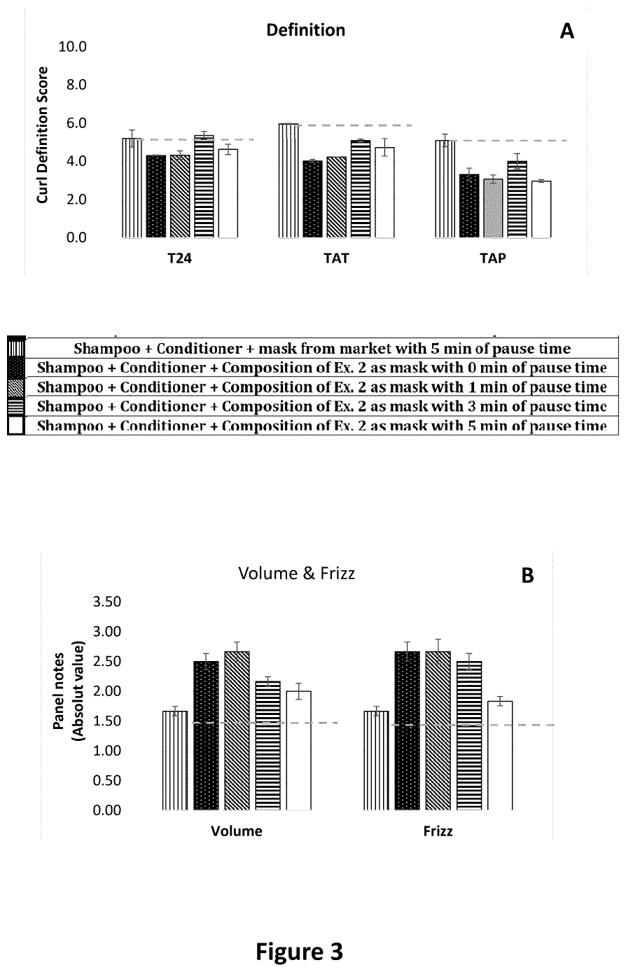

Composition of Ex. 2 and the state-of-the-art composition were applied on virgin curly hair swatches (three 2.7-gram swatches per composition) after the steps of contacting the hair with a shampoo and a conditioner, and were assessed according to a curl definition scoring (from 0 to 10) at 24 hours after treating the hair (T24), at 24 hours but after breaking the rigid effect (passing the fingers through the hair (TAT) and after combing the swatch of TAT (TAP). The definition measurement was done by imaging data. The results are presented on FIG. 3A for several times between application of the composition to the hair and removal from the hair including no time (0 min.), 1 minute between application of the composition to the hair and removal from the hair (1 min.), 3 minutes between application of the composition to the hair and removal from the hair (3 min.), and 5 minutes between application of the composition to the hair and removal from the hair (5 min.).

Additionally, composition of Ex. 2 and the state-of-the-art were applied on virgin curly hair swatches (three 2.7-gram swatches per composition) after the steps of contacting the hair with a shampoo and a conditioner and were assessed according to volume and frizz control. The measurement data was generated by a trained panel and the score goes from 1 to 4 in 0.5 in 0.5 where 4 is the highest score (best value). The results are presented on FIG. 3B.

The data shows that the composition Ex.2 with 3 min of time between application of the composition to the hair and removal from the hair has equal result in immediate definition (T24) as standard routine with 5 min between application of the composition to the hair and removal from the hair but lost in lasting definition (TAT and TAP) (FIG. 3A). In Volume and Frizz control (FIG. 3B), all time between application of the composition to the hair and removal from the hair shows superiority.

1. A rinse-off hair cosmetic composition comprising:
(a) at least one organic acid;
(b) a non-aqueous solvent comprising at least one compound with at least one hydroxyl-group;
(c) at least one amino acid;
(d) at least one fatty compound selected from vegetal oils, vegetal resins, vegetal waxes, and mixtures thereof;
(e) water; and
(f) at least one surfactant selected from cationic surfactants, nonionic surfactants, and mixtures thereof;
wherein the time between application of the cosmetic composition to the hair and removal from the hair ranges from 0 to 10 minutes.

2. The composition according to claim 1,
wherein the organic acid is selected from short chain α-hydroxy acids (AHAs), beta hydroxy acids, and a mixture thereof.

3. The composition according to claim 1, wherein the non-aqueous solvent comprising at least one compound with at least one hydroxyl-group is selected from polyols, C₂ to C₆ monoalcohols, and mixtures thereof.

4. The composition according to claim 1, wherein the at least one amino acid is selected from arginine, glycine, and mixtures thereof.

5. The composition according to claim 1, wherein the at least one fatty compound is selected from: (a) vegetal resins selected from Copaiba officinalis resin, Candeia resin, white breu resin, and mixtures thereof; and/or (b) vegetal oils selected from coconut oil, Soja Soybean oil and mixtures thereof; and/or (c) vegetal waxes selected from Candelilla wax, Carnauba wax, beeswax, and mixtures thereof.

6. The composition according to claim 1, wherein the at least one surfactant includes at least one cationic surfactant is a quaternary ammonium salt selected from cetrimonium chloride, stearimonium chloride, behentrimonium chloride, behentrimonium methosulfate, behenamidopropyltrimonium methosulfate, stearamidopropyltrimonium chloride, arachidtrimonium chloride, distearyldimonium chloride, dicetyldimonium chloride, tricetylmonium chloride, and/or the at least one cationic surfactant is an amino amide cationizable surfactant selected from oleamidopropyl dimethylamine, linoleamidopropyl dimethylamine, isostearamidopropyl dimethylamine, stearamidopropyl dimethylamine, behenamidopropyl dimethylamine, behenamidopropyldiethylamine, behenamidoethyldiethylamine, behenamidoethyldimethylamine, arachidamidopropyldimethylamine, arachidamido-propyidiethylamine, arachidamidoethyidiethylamine, arachidamidoethyidimethylamine, brassicamidopropyl dimethylamine, lauramidopropyl dimethylamine, myristamidopropyl dimethylamine, dilinoleamidopropyl dimethylamine, palmitamidopropyl dimethylamine, dipalmitoylethyl hydroxyethylmonium methosulfate, and mixtures thereof.

7. The composition according to claim 1, wherein the at least one surfactant includes at least one nonionic surfactant selected from at least one fatty alcohol selected from cetearyl alcohol, cetyl alcohol, stearyl alcohol, lauryl alcohol, myristyl alcohol, arachidyl alcohol, behenyl alcohol, an alkoxylated alcohol, and mixtures thereof.

8. The composition according to claim 1, further comprising at least one ester selected from isopropyl palmitate, dicaprylyl carbonate, cetyl esters, and mixtures thereof, wherein the amount of the at least one ester ranges from 0.5% to 10.0% by weight, based on the total weight of the composition.

9. The composition according to claim 1, wherein it further comprises cosmetically acceptable ingredients selected from perfume/fragrance, preserving agents, vitamins, vegetal extracts, pH-adjusters, and mixtures thereof.

10. The composition according to claim 1, wherein it further comprises one or more pigments and/or wherein it is a conditioner or a mask.

11. The composition according to claim 1, wherein:
(a) the at least one organic acid is selected from tartaric acid, lactic acid, glutaric acid, etidronic acid, salicylic acid, glycolic acid, citric acid, and mixtures thereof;
(b) the polyol is selected from propylene glycol, dipropylene glycol, butylene glycol, pentylene glycol, hexylene glycol, glycerin, and mixtures thereof; and/or
(c) the at least one amino acid is arginine.

12. A method of treating hair, comprising the steps of:
(1) contacting the hair with a rinse-off hair cosmetic composition comprising:
(a) at least one organic acid;
(b) a non-aqueous solvent comprising at least one compound with at least one hydroxyl-group;
(c) at least one amino acid;
(d) at least one fatty compound selected from vegetal oils, vegetal resins, vegetal waxes, and mixtures thereof;
(e) water; and
(f) at least one surfactant selected from cationic surfactants, nonionic surfactants, and mixtures thereof;
(2) rinsing off the composition after a period of time.

13. The method according to claim 12, wherein the composition further comprises one or more pigments and/or wherein said time between application of the composition to the hair and removal from the hair ranges from 0 to 10 minutes.

14. A method for providing improved curl definition to the hair, wherein the method comprises contacting the hair with the hair care cosmetic composition according to claim 1.

15. (canceled)

16. The composition according to claim 1, wherein the amount of the at least one organic acid ranges from 0.1% to 25.0% by weight, based on the total weight of the composition.

17. The composition according to claim 1, wherein the amount of the non-aqueous solvent comprising at least one compound with at least one hydroxyl-group ranges from 0.1% to 20.0% by weight, based on the total weight of the composition.

18. The composition according to claim 1, wherein the amount of the at least one amino acid ranges from 0.1% to 7.0% by weight, based on the total weight of the composition.

19. The composition according to claim 1, wherein the amount of the at least one fatty compound ranges from 0.1% to 14.0% by weight, based on the total weight of the composition.

20. The composition according to claim 1, wherein the amount of the at least one cationic surfactant ranges from 0.05% to 15.0% by weight, based on the total weight of the composition.

21. The composition according to claim 1, wherein the amount of the at least one fatty alcohol ranges from 1.0% to 12.0% by weight, based on the total weight of the composition.
